(19) United States
(12) Patent Application Publication
(10) Pub. No.: US 2005/0010191 A1
(43) Pub. Date: Jan. 13, 2005

(54) Essentially tubular body passage lengthening device and method

(76) Inventors: Michael A. Skinner, Chapel Hill, NC (US); Shawn David Safford, Durham, NC (US)

Correspondence Address: MACCORD MASON PLLC, 300 N. GREENE STREET, SUITE 1600, P. O. BOX 2974, GREENSBORO, NC, 27402, US

(21) Appl. No.: 10/852,091

(22) Filed: May 22, 2004

Related U.S. Application Data
(60) Provisional application No. 60/473,049, filed on May 23, 2003.

(51) Int. Cl. A01N 59/08; A61M 31/00

(52) U.S. Cl. 604/500

(57) ABSTRACT

An intestinal lengthening device and method for lengthening the intestine of infants and neonates. The method steps include: resecting a section of intestine to be lengthened; closing the one end of the intestine section, preferably the proximal end, and attaching the other end to an opening in the abdominal wall to form a fistula with stoma; allowing the attachments to heal; attaching the intestinal lengthening device; applying and maintaining 5-20 kPa of tension to the device; allowing the gut to rest for 7 days when the desired length is reached; removing the intestinal lengthening device; reattaching the intestinal section to the intestine. The device includes an inserted end that attaches to the mucosal surface of an intestinal section, an abdominal end that attaches to the abdomen of the patient, and a tension rod to incrementally increase the distance between the two ends. An in situ device includes a sealed tension rod between 2 expansion tips.

This nonprovisional utility patent application claims the benefit of a prior filed provisional application: 60/473,049 filed May 23, 2003, which is incorporated herein by reference in its entirety.

### BACKGROUND OF THE INVENTION

(1) Field of the Invention

The present invention relates generally to surgical instruments and methods and, more particularly, to a device and method for lengthening intestines.

(2) Description of the Prior Art

Short gut syndrome currently affects 10,000-20,000 children in the United States. Causes of short gut include malrotation and necrotizing enterocolitis. Currently, the treatment for short gut syndrome consists of long-term total parenteral nutrition and various operative techniques that are temporizing at best. One method to increase length of esophageal atresias includes lengthening by static force via sutures through the chest wall. Clinical evidence suggests that mechanical tension will stimulate lengthening and growth of the alimentary tract in infants and neonates.

Prior art devices have used grips that attached on the serosal side of the gut by suturing (Small Bowel Lengthening by Mechanical Distraction. Printz et al., Digestion 1997; 58:240-248). The device used a serosal grip, which requires that the device be retained in the serosal cavity. To achieve significant lengthening of a section of gut, the prior art device would require that a long tension rod be internalized inside the patient. The need for continual increasing tension requires that the physician invade the serosal space on a daily basis, if not more frequently. This frequent invasion puts the patient at risk for infection. Additionally, the authors stated that no significant increase in overall gut length was observed (p. 245).

Another prior art method of intestinal lengthening involves using hydraulic pressure to increase gut length. As described in An Animal Experiment on Short Gut Lengthening Chen, Y. et al. Chinese Medical Journal 110: 5: 354-357 (1997), New Zealand white rabbits were used to test the efficacy of hydraulic pressure as an intestinal-lengthening force.

A 5 cm segment of the terminal ileum with a pedicle was put underneath the abdominal wall, with its proximal end closed and its distal end exteriorized as a stoma. One week later, as the wound healed well, an expander made of a small rubber tube was inserted into the short gut loop through the stoma. Three milliliters of water was injected as an initial dose. Then 0.5 ml water as an increment was injected subsequently every 12 hours until the total amount of water reached 15 ml and kept stable for two weeks. Results: Anatomically, the length and the capacity of the intestinal loop were recorded, after 15 ml expanding, as an average of 150% lengthening of the original and an average of 293% expansion. Two weeks after the removal of the expander, partial shrinking was observed. A stable lengthening had 123% of the original length, while the capacity reduced to 200% of the original one. Histologically, there were no remarkable changes of the mucosa or submucosa, but significant hypertrophy of the musculature and serosa layer was observed. The total thickness of the musculatures was 618% of the normal controls'. Ultrastructurally, there were enlargement of the smooth muscle cells, increase in number and size of the mitochondria in the cytoplasm and widening of the intercellular space. The results indicate that hydraulic pressure can be used to lengthen a section of intestine; however, the intestine also expands in circumference, which is an undesirable result because it produces a decreased surface-to-volume ratio, which decreases the efficiencies of absorption of nutrients.

Thus, there remains a need for an intestinal lengthening method and device that is easily adjustable externally without putting the patient at continuous risk of infection during adjustment and that does not expand the intestine in circumference.

### SUMMARY OF THE INVENTION

The present invention is directed to an intestinal lengthening method and device that is easily adjustable externally without putting the patient at continuous risk of infection during adjustment and that does not expand the intestine in circumference.

In a preferred embodiment, a mechanical extension method and device are provided. More preferably, the method and device are employed in vivo, thereby providing continued use of the intestinal region to be extended during its extension, which prevents atrophy and improves recovery time for the patient.

The present invention is further directed to a method for mechanically extending a predetermined section of intestinal tract, more particularly, the small intestines.

Thus, the present invention provides both a method and device for mechanically extending a predetermined section of intestinal tract to improve nutrient absorption through increased length, which is maintained after removal of the device.

These and other aspects of the present invention will become apparent to those skilled in the art after a reading of the following description of the preferred embodiment when considered with the drawings.

### BRIEF DESCRIPTION OF THE DRAWINGS

FIG. 1 is a side view of an intestinal lengthening device constructed according to the present invention.

FIG. 2 is the device of FIG. 1 installed in a section of intestine.

FIG. 3 is a side view of another embodiment constructed according to the present invention.

FIG. 4 is a close-up perspective view of the inserted end of the embodiment shown in FIG. 3.

FIG. 5 is an end view of the undeployed inserted end of the embodiment of FIG. 3.

FIG. 6 is a close-up perspective view of the inserted end of the embodiment shown in FIG. 4 in a deployed mode.

FIG. 7 is an end view of the deployed inserted end of the embodiment of FIG. 3.

FIG. 8 shows side views of a completely insertable device in 3 states.

FIG. 9 is a cut-away side view of a magnetostrictive expansion tip in 2 states.

FIG. 10 is a cut-away side of a magnetostrictive device with SFS sensor in 3 states.

FIG. 11 is a side view of a thin-film, bimorph cantilever magnetostrictive prong in 3 states.

FIG. 12 is a side view of a device with magnetostrictive tension component, SFS sensor, and magnetostrictive prongs in 3 states.

### DETAILED DESCRIPTION OF THE PREFERRED EMBODIMENTS

In the following description, like reference characters designate like or corresponding parts throughout the several views. Also in the following description, it is to be understood that such terms as “forward,” “rearward,” “front,” “back,” “right,” “left,” “upwardly,” “downwardly,” and the like are words of convenience and are not to be construed as limiting terms. Because it may be confusing to describe the interior and exterior of the intestine, the terms “mucosal” and “serosal” are used to distinguish the surfaces of the intestine.

Referring now to the drawings in general, the illustrations are for the purpose of describing a preferred embodiment of the invention and are not intended to limit the invention thereto. The present invention provides a mechanical extension method and device, which are preferably employed in vivo, to extend at least one predetermined section of intestinal tract, more particularly, the small intestines. In one embodiment of the present invention, the device and method further provide for continued use of the intestinal region to be extended during its extension, which prevents atrophy and improves recovery time for the patient. Alternatively, the intestinal region to be extended may be resected from the small intestine of the patient; the remaining intestine (not shown) is connected to allow intestinal contents to continue passing.

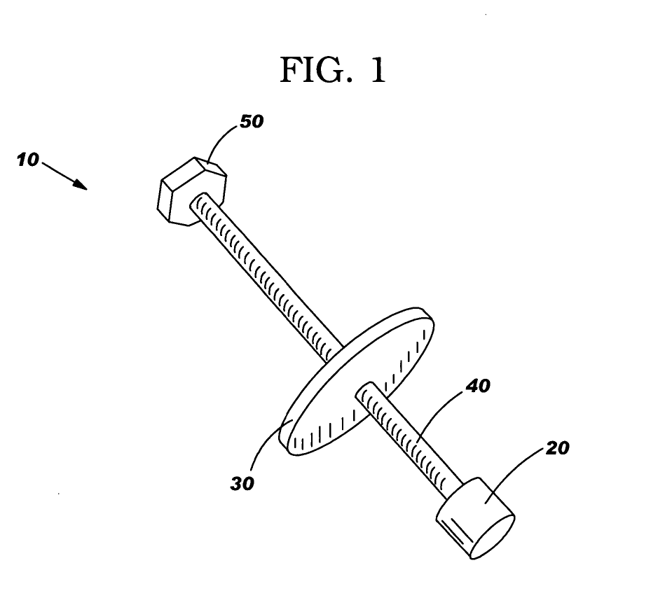

As best seen in FIG. 1, the present invention, generally referred to as 10, includes an inserted end 20, an abdominal end 30, and a tension rod 40. The device is designed to provide incremental changes in distance between the ends 20 and 30. In a preferred embodiment of the intestinal extension device of the present invention, the tension rod is a threaded rod and the abdominal end 30 is threaded internally to receive the threaded tension rod and allow it to pass through. Turning the tension rod free end 50 increases or decrease the distance between the two inserted end and the abdominal end. The tension rod free end 50 is appropriately shaped to facilitate turning; for example, it can be non-circular, such as hexagonal or square, to receive a wrench. The inserted end 20 is designed to spread the pressure exerted on the gut during lengthening over a sufficient area such that the end of the intestine is not damaged or punctured by excessive pressure.

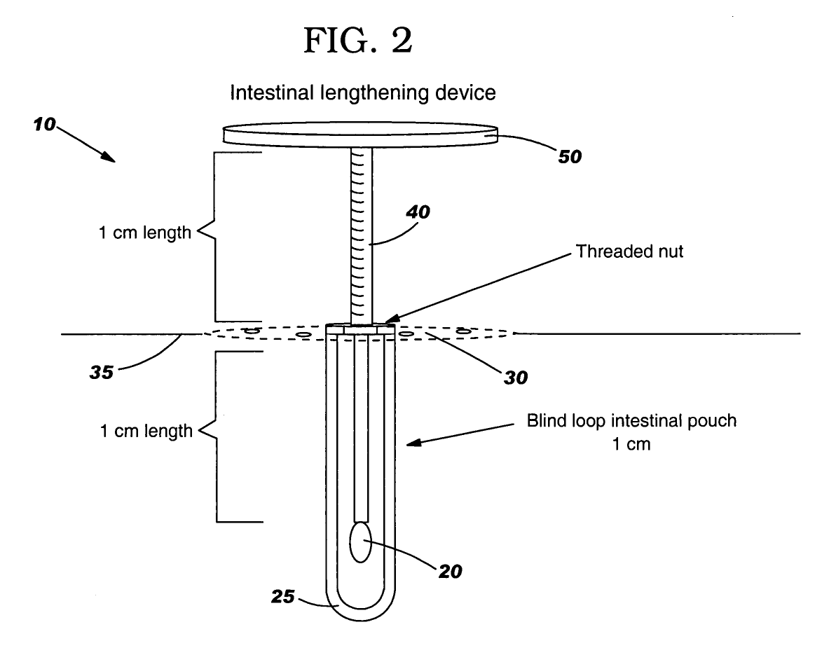

FIG. 2 shows the present invention installed in a section of intestine. The intestinal section is resected from the small intestine of the patient. One end is closed 25 by suturing or other appropriate means; the other end is sutured to the abdominal wall 35, forming a stoma or fistula. The remaining intestine (not shown) is connected to allow intestinal contents to continue passing. The sutured connections are allowed to heal, approximately 1 week, prior to the application of tension. The tension is applied by turning the external tension rod end 50 until sufficient tension is applied. The rod is advanced 1 mm per day, which has been shown in in vitro experiments to apply approximately about 5 to about 10 kPa of tension to stimulate intestinal lengthening. The tension is maintained until the desired amount of intestinal lengthening is achieved. Thereupon, the device is no longer lengthened but left attached for one week such that the intestinal section can finish adapting to the remaining tension.

A second embodiment would also include creating a blind loop ostomy through suturing the mucosal surface of the intestine onto itself to create an intraluminal buttress. This would allow tension to be created and after a segment of bowel has been lengthened by about 2-3 cm, the suture would be removed and the device would be retracted. Once the device was retracted, a new suture would be placed in the proximal bowel. The device would then be redeployed against this new area and the bowel would continue to be lengthened segmentally. This would be repeated until an adequate length of bowel would be created.

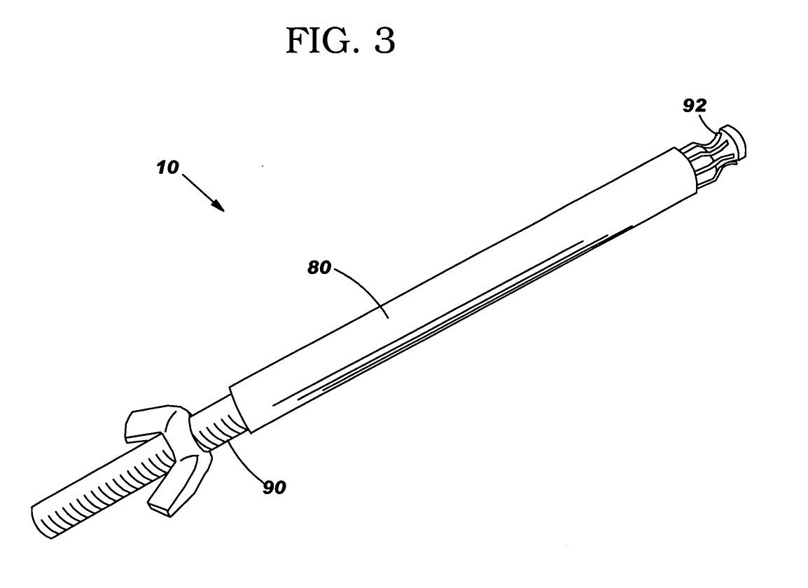

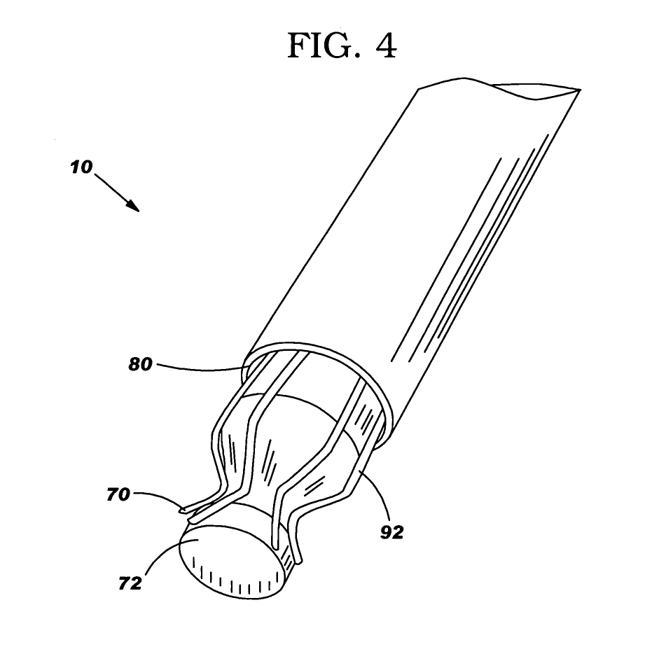

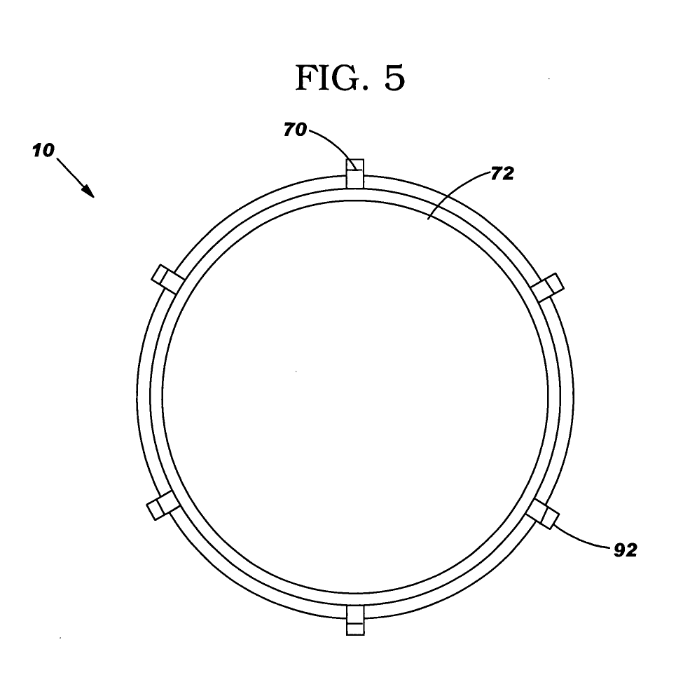

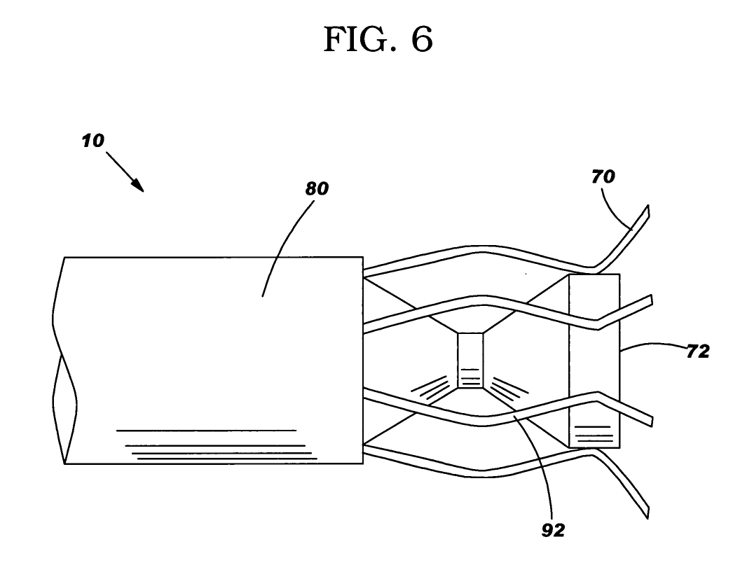

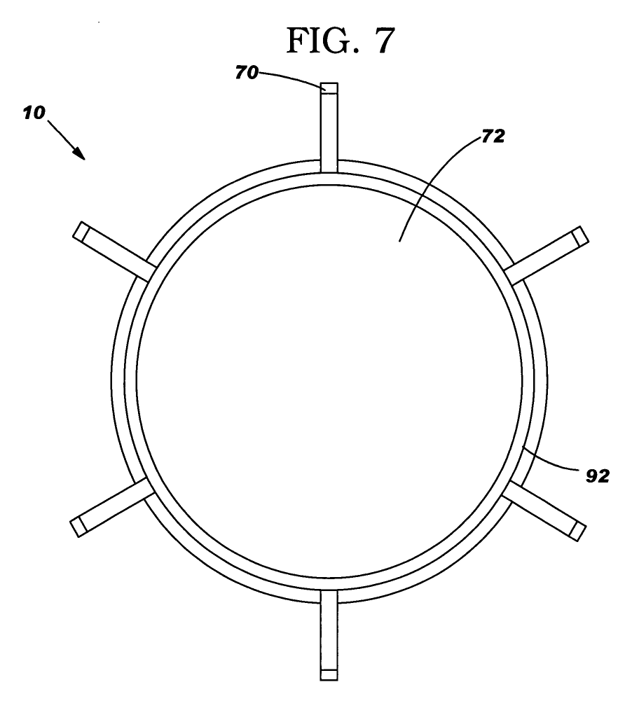

An alternative embodiment of the present invention is shown in FIGS. 3 through 7. In these figures, the intestinal extension device 10 includes a tubular body portion 80 having a screw portion 90 inserted therein and corresponding to matching threaded interior surface region (not shown) of the body portion for applying an extending/lengthening force at a tip end 92 which is inserted into the intestines to be lengthened. As seen in FIGS. 4 and 5, the inserted tip end 92 includes an expansion tip 72 around which are arrayed a plurality of prongs 70 that are expanded by retraction or extension of the expansion tip after insertion of the device into a predetermined section of the intestines to be lengthened to provide for a distribution of the mechanical extending force applied by the device upon deployment, which is shown in FIGS. 6 and 7. The prongs are fixed to the interior of the body portion 80, whereas the expansion tip is movable with respect to the body portion.

The expansion tip is shaped to prevent the prongs from catching on the mucosal surface during insertion in the intestine but prior to deployment. Thus, the expansion tip can have an hour-glass shape, as shown in FIGS. 4, 6, and 8, which allows the prongs to recess into the expansion tip when not deployed. When the expansion tip is moved in either direction from the resting position relative to the body portion 80, it flexes the prongs outward.

The prongs 70 are designed to expand into the intestinal wall and prevent the device from traveling further in the intestine in the direction of the prongs. The end shape, length, and flexibility of the prongs and prong material are such that the exerted expansion force transmitted through the prongs to the intestinal wall does not exceed about 20 kPa when the expansion tip is expanded to its full size, thereby preventing puncturing of the intestinal wall by a prong. The mucosal surface of the intestine is sufficiently irregular that a prong with an end diameter of about 0.25 mm expanded into the intestine with a force of about 5-20 kPa will not slip down the intestine when a distraction force of about 10 kPa is applied to the device. The number of prongs is sufficient to hold the device in place and to perform other relevant functions. Preferably, about 4 to about 10 prongs are used on a tip. For the current working model, the device in a non-deployed state measures 10 mm across and in the deployed state measures 18 mm.

The prongs are designed such that intestinal contents can flow past them when the prongs are deployed. As shown in FIG. 7, the prongs have a narrow cross-section and extend beyond the exterior diameter of the tubular body portion 80, thereby permitting intestinal contents to flow past them and between the intestinal wall and the tubular body portion. Thus, the present invention permits deployment in an intact intestine in situ without resection of a segment and allows the section being distracted to continue its absorptive and other functions.

FIG. 4 is a close-up perspective view of the inserted end of the embodiment shown in FIG. 3. FIG. 5 is an end view of the undeployed inserted end of the embodiment of FIG. 3. FIG. 6 is a close-up perspective view of the inserted end of the embodiment shown in FIG. 4 in a deployed mode. FIG. 7 is an end view of the deployed inserted end of the embodiment of FIG. 3.

This device would be placed through an external ostomy that would be created at an initial surgery whereby a segment of bowel would be resected and continuity would be created through anastomosing the distal segment to 2-3 cm proximal to the ostomy limb. Once in place, the device would be placed through the ostomy and the device deployed to grasp the intestine. Once deployed, the device would be advanced through a screw mechanism at the proximal end of the device. The device would be advanced at a rate of 1-2 mm per day, not to exceed approximately 20 kPa of stress on the intestinal wall, as measured by an external monitor.

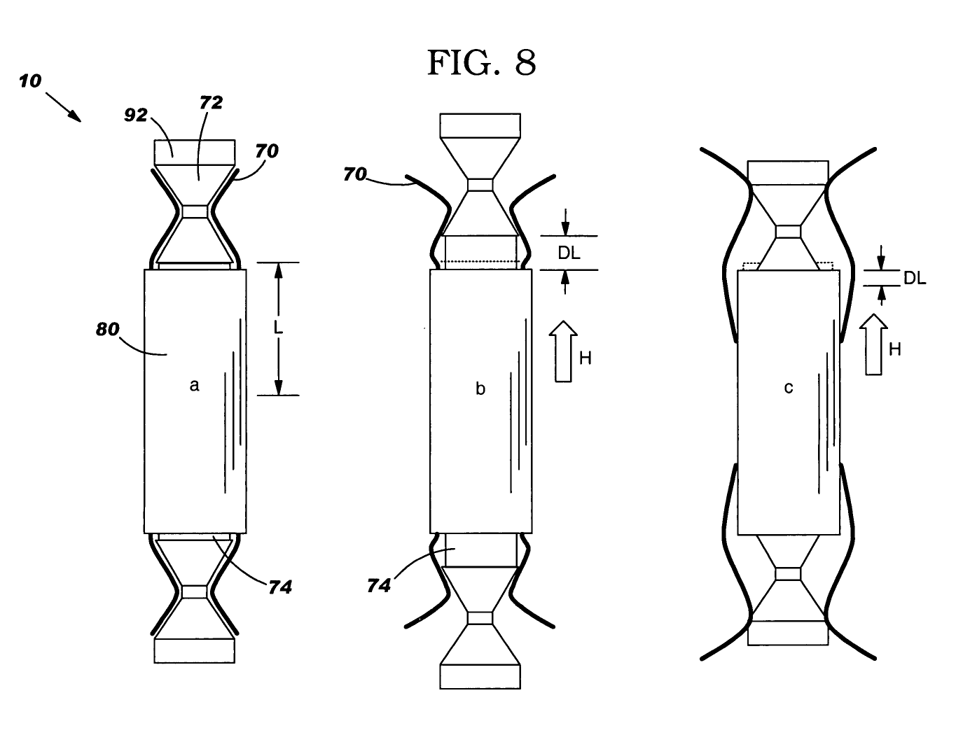

Another preferred embodiment according to the present invention is a completely insertable device 100, as shown in FIG. 8. The completely insertable device incorporates an expansion tip 72 at the 2 longitudinal ends and a sealed tension rod 74 between the 2 expansion tips. The expansion tips work in opposition, providing resistance to one another when expanded and the sealed tension rod is lengthened. The expansion tips includes prongs, as previously described, which deploy when the tension rod is initially lengthened.

The sealed tension rod can be activated using an endoscope with mechanical actuator tip or can be activated remotely.

A magnetostrictive microactuator can be used to provide the expansion and distraction force to the opposing expansion tips. Magnetostriction is the change in dimension of a magnetic material under applied magnetic fields. A negative magnetostriction is the contraction of the material along the magnetic field and its expansion in the perpendicular direction, whereas a positive magnetostriction is the reverse of these movements.

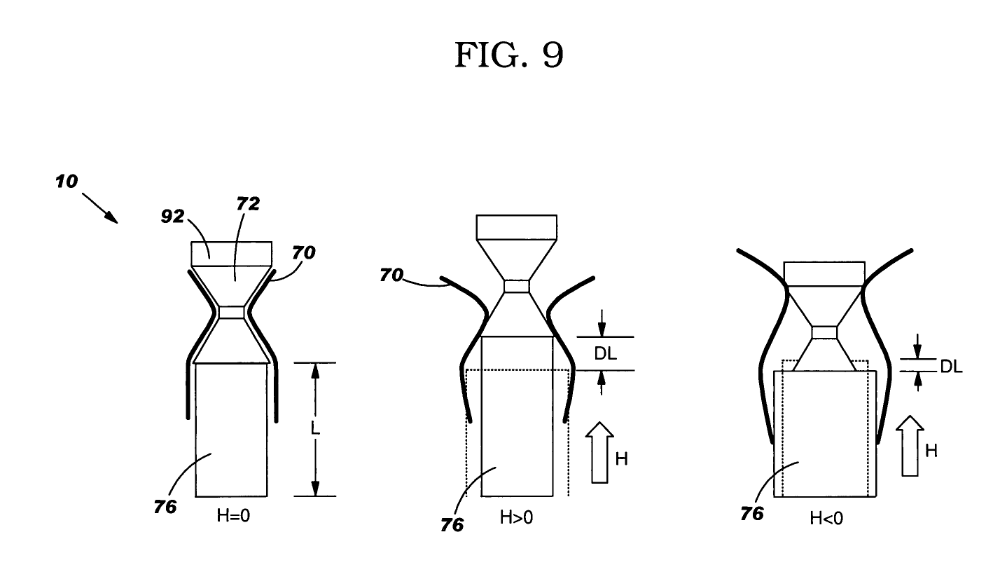

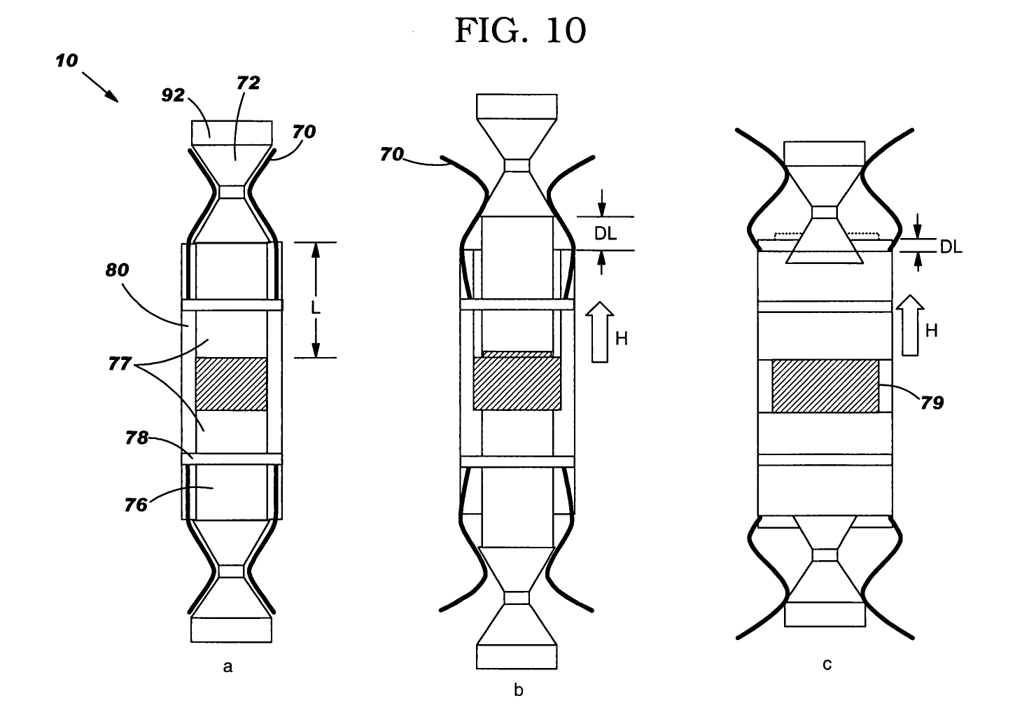

FIG. 9 shows a cut-away view of one functional end of completely insertable device wherein the lengthening function is performed by a magnetostrictive microactuator 76. In this figure, H symbolizes a magnetic field applied to the magnetostrictive material. The influence on the shape of the material under three different conditions: from left to right, there is no field, so the material is at its original shape, then a positive field is applied and the material expands, finally, the field is negative and the material retracts. FIG. 10 shows a cut-away view of a completely insertable device. In this device, each functional end as a first magnetostrictive device 76, and intervening base plate 78, and a second magnetostrictive device 77. The prongs are attached to the intervening base plate. When the first magnetostrictive device 76 is activated, the expansion tip forces the prongs outward, deploying them. When the second magnetostrictive device 77 is activated, the prongs are forced along the longitudinal axis of the device, exerting a lengthening force on the intestine.

A ratchet mechanism (not shown) can be used to extend the effective range of expansion of the device. The ratchet mechanism can turn a screw-based tension rod. Alternatively, the device can be operated discontinuously. That is, the device can be activated and maintained in an expanded state until the tension on the device falls below a predetermined level due to the lengthening of the intestine, at which time the device is then contracted and re-expanded. The device may be moved to a new location or may be re-expanded in the same location. Once it is desired to remove the device from the body, the device is simply retracted and allowed to pass out with the intestinal contents.

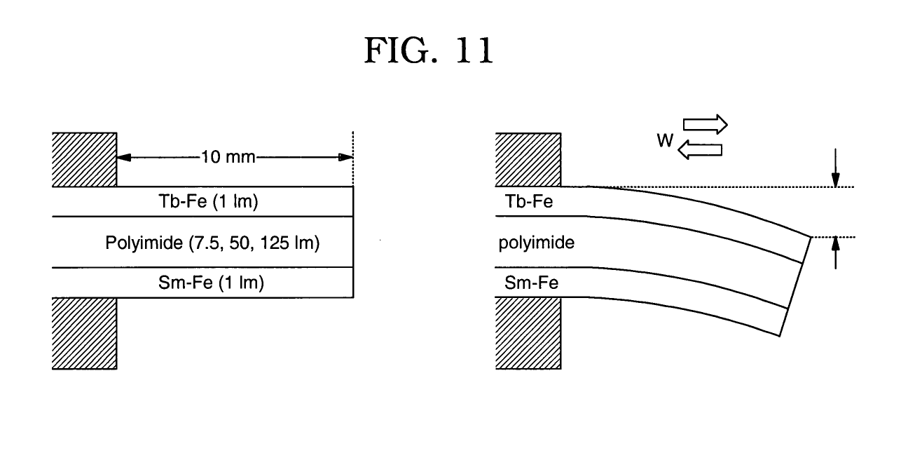

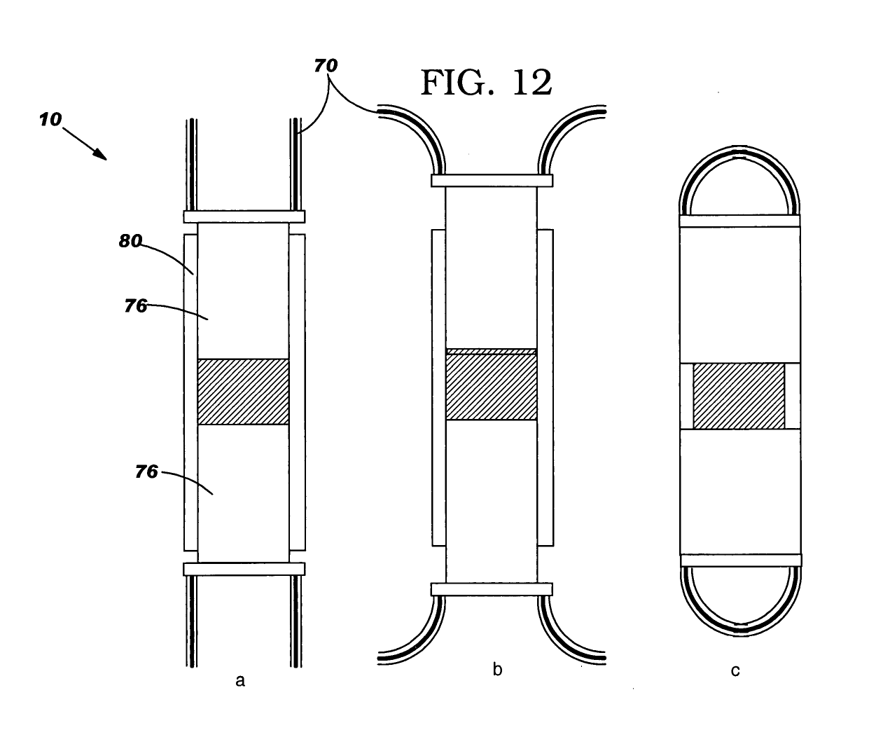

Additionally, the magnetostrictive effect can be used in the prongs to provide a thin-film bimorph cantilever, shown as a side view in FIG. 11. The deflection of the prongs in this manner reduces the length increase of the device lost during deployment of the prongs. In this case a single magnetostrictive device 76 can be used; the prongs are attached this device and are activated prior to activation of the magnetostrictive component. FIG. 12a shows the device in neutral position; FIG. 12b shows the device with prongs deployed and magnetostrictive device expanded; FIG. 12c shows the device with prongs and magnetostrictive device retracted for passage through the intestine.

There are multiple means for measuring the tension on the tension rod. Without limiting the means available, the tension on the tension rod can be monitored with a MEMS device, such as a strain and force sensor (SFS). An SFS measure a force on a solid. There are two types of SFS: quantitative and qualitative. Quantitative Strain and Force Sensors, such as strain gauges and load cells, assess the force and proportionally represent its value into an electric signal. Qualitative Strain and Force Sensors are a Boolean type of output signal and do not represent the force value as accurately. They detect if there is a sufficient force applied and the output signal indicates when the predetermined threshold is reached. The signal generated by the SFS is then transmitted to an external reader, which then determines the stress on the device. FIG. 10 shows a device with a SFS sensor 79 positioned between the two second magnetostrictive devices that cause the device to lengthen. Lengthening of the device will cause static force to be exerted on the SFS sensor if the prongs are inserted in the intestine correctly, thereby holding their position and not slipping. As the gut lengthens, the static force on the SFS will diminish. The SFS can signal this diminished force to the operator, who then can manipulate the device to increase the force or move the device to a new location.

The completely insertable device and the intestine in the vicinity of the device can be monitored using an endoscope or with the method described in U.S. Pat. No. 6,514,082 issued to Kaufman, et al. Feb. 4, 2003 for System and method for performing a three-dimensional examination with collapse correction, incorporated herein by reference in its entirety. U.S. Pat. No. 6,514,082 provides a method for navigating through an organ, such as a colon, via a computer generated image of the organ.

A multiplicity of completely insertable intestinal lengthening devices thus described can be simultaneously inserted and deployed in a single patient, thereby allowing for a rapid increase in total gut length.

Experimental Results

The present invention was tested in a series of experiments in rats. In the first round of experiments, 125 weaned rat pups at 28 days were subject to intestinal lengthening with the embodiment shown in FIG. 1. Weaned rat pups were used as these have historically been used for intestinal experiments, the size capability of the intestines is proven to hold suture, and clinical evidence to suggest that mechanical tension will stimulate the lengthening and growth of the alimentary tract has only been reported in infants and neonates.

In the first experiment the tension rod was advanced about 1 mm daily to create a daily increase in tension. Juvenile rats underwent midline laparotomies where an average 15 mm segment of bowel was isolated. A double-barreled, blind-loop ostomy was created using the isolated segment of bowel. Our intestinal lengthening device was inserted into one of the loops and the second loop was used as an internal control for our experiment. As a control for normal growth of the intestine, a 1 cm length of in situ bowel was marked. After recovery, the lengthening device was advanced approximately 1 mm/day. After 30 days, the animals were sacrificed. Tissues examined included the lengthened segment of bowel, the control from the unlengthened segment of bowel, and the in situ segment of bowel. The intestinal lengthening device increased the length of bowel an average of 220% in comparison to the control unlengthened segment of bowel (p<0.001). No difference was identified between the in situ bowel and the unlengthened bowel (p=0.4). The lengthed loop of bowel showed no difference in total mucosal thickness, villous height, or villous width in comparison to in situ bowel (p=0.3). Total mucosal thickening was increased in the lengthened loop of bowel (37.2 μm v. 5.8 μm, p<0.001). We measured enzyme activity of bowel as an indirect measurement of functionality. Enzyme activity was increased in the lengthened limb of bowel to over twice in all enzymes measured that included sucrase, lactase, maltase, and palatinase. This is good evidence for not only increase in length, but increase in function.

Thus, a method for lengthening the intestine of infants and neonates includes the following steps:

1) resecting a section of intestine to be lengthened;

2) closing the one end of the intestine section, preferably the proximal end, and attaching the other end to an opening in the abdominal wall to form a fistula with stoma;

3) allowing the attachments to heal;

4) attaching the intestinal lengthening device;

5) applying and maintaining about 5 to about 10 kPa of tension to the device;

6) allowing the gut to rest for 7 days when the desired length is reached;

7) removing the intestinal lengthening device;

8) reattaching the intestinal section to the intestine.

Alternatively, the method may include the steps:

1) division of the bowel;

2) suturing the distal segment of bowel 2-4 cm proximal to the proximal limb to create normal continuity of the bowel;

3) creating an ostomy with the proximal limb;

4) suturing the lumen of the bowel with full serosal stitches to create a narrowing in order to apply tension to the limb of bowel;

5) the screw device would be deployed against the narrowed limb;

6) applying and maintaining about 5 to about 10 kPa of tension to the device to advance 2-4 cm;

7) retracting the deivce;

8) resuturing of the bowel at the opening of the ostomy;

9) redeploying of the device;

10) repeating steps 4, 5, 6, 7, 8 and 9 until sufficient gut length is generated;

11) allowing the gut to rest for 7 dayse when the desired length is reached;

12) removing the intestinal lengthening device;

13) reattaching the intestinal section to the intestine

Alternatively, the method may include steps that provide for resecting the predetermined section of the intestines to be lengthened, inserting the device for mechanical lengthening thereof, and maintaining the section of the intestines to be lengthened/extended within the nutrient flow to permit the absorption of nutrients thereby during the lengthening process.

For the in situ devices, the method of employment includes:

1) inserting the device in the intestine;

2) deploying the prongs;

3) lengthening the tension component;

4) retracting the prongs and tension component when the distraction is complete;

5) repeating steps 2, 3, and 4 until sufficient gut length is generated;

6) retracting the prongs and tension component when the total distration is complete;

7) allowing the device to pass through the intestines.

Certain modifications and improvements will occur to those skilled in the art upon a reading of the foregoing description. All modifications and improvements have been deleted herein for the sake of conciseness and readability but are properly within the scope of the following claims.

1. A lengthening device for extending essentially tubular body passages such as the intestine, esophagus, ureter, and the like, and combinations thereof, comprising
a) a tension rod having a free end and an inserted end,
b) a force distributor, and
c) an anchor
wherein the force distributor being connected to the inserted end of the tension rod and the tension rod is passed through the anchor,
the force distributor being placed inside a segment of an essentially tubular body passage that has been resected, closed at one end, and attached at the non-closed end to an abdominal wall, and
manipulating the tension rod causes incremental changes between the force distributor and the anchor
thereby lengthening a essentially tubular body passage segment without decreasing the circumference of the segment.

2. The lengthening device according to claim 1, wherein the tension rod is a threaded rod.

3. The lengthening device according to claim 1, wherein the tension rod free end is appropriately shaped to facilitate turning.

4. The lengthening device according to claim 1, wherein the tension rod free end is non-circular.

5. The lengthening device according to claim 1, wherein the tension rod free end is hexagonal-osquare-shaped.

6. The lengthening device according to claim 1, wherein the tension rod free end can receive a wrench.

7. The lengthening device according to claim 1, wherein the anchor is threaded internally to receive the threaded tension rod and allow it to pass through.

8. The lengthening device according to claim 1, wherein the force distributor is designed to spread the pressure exerted on the essentially tubular body passage such that the essentially tubular body passage is not damaged or punctured by excessive pressure.

9. The lengthening device according to claim 1, wherein turning the tension rod free end changes the distance between the inserted and abdominal ends.

10. A method for lengthening an essentially tubular body passage, such as the intestine, esophagus, ureter, and the like, and combinations thereof, comprising the steps of:
a) resecting a segment of an essentially tubular body passage,
b) closing one end of the segment,
c) attaching the open end of the segment to the abdominal wall thereby forming a stoma or fistula,
d) allowing the sutured connections to sufficiently heal before applying tension,
e) applying tension by using the lengthening device according to claim 1, and
f) maintaining tension until the desired amount of essentially tubular body passage lengthening is achieved.

11. The method of claim 10, further comprising the step of reconnecting the non-resected essentially tubular body passage to allow the essentially tubular body passage contents to continue passing.

12. The method of claim 10, wherein the segment of the essentially tubular body passage is resected from the small intestine.

13. The method of claim 10, further comprising the steps of
closing one end of the essentially tubular body passage segment with sutures, and
attaching the open end of the essentially tubular body passage segment to the abdominal wall with sutures.

14. The method of claim 10, further comprising the steps of
applying tension by manipulating the tension rod until sufficient tension is applied to the essentially tubular body passage segment, and
advancing the tension rod 1 mm per day
thereby providing approximately 5-10 kPa of stress to stimulate essential tubular body passage lengthening.

15. The method of claim 10, wherein the time period to allow the sutured connections to sufficiently heal is approximately one week.

16. The method of claim 10, further comprising the steps of
no longer manipulating the tension rod once the desired amount of essentially tubular body passage lengthening is achieved and
leaving the lengthening device attached for sufficient time such that the essentially tubular body passage section can finish adapting to the remaining tension.

17. The method of claim 16, wherein the sufficient time such that the essentially tubular body passage section can finish adapting to the remaining tension is one week.

18. A method for lengthening an essentially tubular body passage, such as the intestine, esophagus, ureter, and the like, and combinations thereof, comprising the steps of:
a) creating a blind loop ostomy through suturing the mucosal surface of the intestine onto itself to create an intraluminal buttress,
b) creating tension by using the lengthening device according to claim 1,
c) removing the suture and retracting the lengthening device after a segment of bowel has been lengthened by approximately 2-3 cm,
d) placing a new suture in the proximal bowel,
e) redeploying the lengthening device against the newly sutured area, and
f) repeating steps a through e until an adequate length of bowel had been lengthened.

19. A lengthening device for extending essentially tubular body passages such as the intestine, esophagus, ureter, and the like, and combinations thereof, comprising
a) a tubular body,
b) a tension rod having a free end and a tip end,
c) an expansion tip, and
d) a plurality of prongs
wherein the tension rod is inserted through the tubular body,
the tension rod tip end is connected to the expansion tip,
the prongs are fixed to the tubular body and surround the expansion tip,
the expansion tip is movable with respect to the tubular body,
the tension rod tip end is inserted into an essentially tubular body passage to be lengthened,
the prongs are expanded by retraction or extension of the expansion tip after the device is inserted into the essentially tubular body passage, and
applying through the tension rod a lengthening force at the tension rod tip end on the essentially tubular body passage
thereby lengthening an essentially tubular body passage segment without decreasing the circumference of the essentially tubular body passage segment,
distributing the mechanical lengthening force applied by the device upon prong expansion,
allowing the essentially tubular body passage contents to flow past the prongs and between the essentially tubular body passage wall and the tubular body, and
deploying the lengthening device in an intact intestine or esophagus in situ without resection and allowing the section being distracted to continue its adsorptive and other functions.

20. The lengthening device according to claim 19, wherein the tubular body has a threaded interior surface region.

21. The lengthening device according to claim 19, wherein the tension rod is a threaded rod.

22. The lengthening device according to claim 19, wherein the expansion tip is shaped to prevent the prongs from catching on the mucosal surface during insertion in the essentially tubular body passage.

23. The lengthening device according to claim 19, wherein the expansion tip has an hourglass shape, such that the prongs may recess into the contours of the expansion tip when not deployed and when the expansion tip is moved in either direction from the resting position relative to the tubular body, the prongs are flexed outward.

24. The lengthening device according to claim 19, wherein the prongs are designed to expand into the essentially tubular body passage wall and prevent the device from traveling further in the essentially tubular body passage in the direction of the prongs.

25. The lengthening device according to claim 19, wherein the shape, length, and flexibility of the prongs and prong material are such that the exerted expansion force transmitted through the prongs to the essentially tubular body passage wall does not exceed approximately 20 kPa when the expansion tip is expanded to its full size, thereby preventing puncturing of the essentially tubular body passage wall by a prong.

26. The lengthening device according to claim 19, wherein the number of prongs is sufficient to hold the device in place and to perform other relevant functions.

27. The lengthening device according to claim 19, wherein the number of prongs is between 4 and 10.

28. The lengthening device according to claim 19, wherein the prongs are designed such that the essentially tubular body passage's contents can flow past them when the prongs are deployed.

29. The lengthening device according to claim 19, wherein the prongs have a narrow cross-section and extend beyond the exterior diameter of the tubular body portion.

30. A method for lengthening an essentially tubular body passage, such as the intestine, esophagus, ureter, and the like, and combinations thereof, comprising the steps of:
a) creating an external ostomy at an initial surgery whereby a segment of the essentially tubular body passage would be resected and continuity would be created through anastomosing the distal segment to the ostomy limb,
b) placing lengthening device according to claim 19 through the external ostomy,
c) deploying the lengthening device to grasp the essentially tubular body passage wall, and
d) advancing the lengthening device to lengthen the essentially tubular body passage.

31. The method of claim 30, wherein continuity would be created through anatomosing the distal segment to 2-3 cm proximal to the ostomy limb.

32. The method of claim 30, wherein advancing the lengthening device is done with a screw mechanism at the proximal end of the device.

33. The method of claim 30, wherein the advancing rate is 1-2 mm per day, not to exceed 20 kPa of stress on the intestinal wall.

34. A lengthening device for extending essentially tubular body passages such as the intestine, esophagus, ureter, and the like, and combinations thereof, comprising
a) a tubular body,
b) a rod-like tension component having two functional ends at the opposite longitudinal ends of the tension component,
c) two expansion tips, and
d) a plurality of prongs
wherein the tension rod is sealed within the tubular body,
each expansion tip is attached to one of the functional ends of the tension component,
the prongs are attached to the functional ends of the tension component and surround the expansion tips,
the expansion tips work in opposition, providing resistance to one another when expanded and the sealed tension component is lengthened,
the tension rod tip end is inserted into an essentially tubular body passage to be lengthened,
the prongs are expanded when the tension component is initially lengthened after the device is inserted into the essentially tubular body passage, and
applying through the tension rod a lengthening force at the tension rod tip end on the essentially tubular body passage
thereby permitting the lengthening device to be completely insertable within the essentially tubular body passage.
lengthening an essentially tubular body passage segment without decreasing the circumference of the essentially tubular body passage segment,
distributing the mechanical lengthening force applied by the device upon prong expansion,
allowing the essentially tubular body passage contents to flow past the prongs and between the essentially tubular body passage wall and the tubular body, and
deploying the lengthening device in an intact essentially tubular body passage in situ without resection and allowing the section being distracted to continue its adsorptive and other functions.

35. The lengthening device according to claim 34, wherein the tension component can measure tension with a MEMS device.

36. The lengthening device according to claim 35, wherein the MEMS device is a strain and force sensor.

37. The lengthening device according to claim 36, wherein the strain and force sensor is a quantitative or qualitative sensor.

38. The lengthening device according to claim 35, wherein the MEMS device transmits a signal to an external reader, which then determines the tension in the lengthening device.

39. The lengthening device according to claim 35, wherein the MEMS device is located within the tension component.

40. The lengthening device according to claim 34, wherein the tension component can be activated using an endoscope with mechanical actuator tip.

41. The lengthening device according to claim 34, wherein the tension component can be activated remotely.

42. The lengthening device according to claim 34, wherein a magnetostrictive microactuator can provide the expansion and distraction force to the opposing functional ends of the tension component.

43. The lengthening device according to claim 34, wherein the tension component can be activated with a magnetic field.

44. The lengthening device according to claim 34, wherein the tension component further comprises
a) a first magnetostrictive device at each functional end,
b) an intervening base plate at each functional end, and
c) a second magnetostrictive device
wherein each first magnetostrictive device is attached to the intervening base plate at each functional end
the prongs are attached to the intervening base plate,
each intervening base plate is connected to the second magnetostrictive device,
activation of the first magnetostrictive device forces the prongs outward, deploying them toward the mucosal wall of the essentially tubular body passage, and
activation of the second magnetostrictive device forces the prongs along the longitudinal axis of the lengthening device
thereby exerting a lengthening force on the essentially tubular body passage.

45. The lengthening device according to claim 34, wherein the tubular body is threaded internally.

46. The lengthening device according to claim 34, wherein the tension component is a threaded rod.

47. The lengthening device according to claim 46, wherein a ratchet mechanism turns the threaded rod, thereby extending the effective range of expansion of the lengthening device.

48. The lengthening device according to claim 34, wherein each prong is a magnetostrictive thin-film bimorph cantilever.

49. The lengthening device according to claim 34, wherein the tension component is a single magnetostrictive device.

50. A method for lengthening an essentially tubular body passage, such as the intestine, esophagus, ureter, and the like, and combinations thereof, comprising the steps of:
a) inserting at least one lengthening device according to claim 34 in the essentially tubular body passage,
b) deploying the prongs,
c) lengthening the tension component,
d) retracting the prongs and tension component when the distraction is complete,
e) repeating steps b, c, and d until sufficient essentially tubular body passage length is generated,
f) retracting the prongs and tension component when the total distraction is complete, and
g) allowing the device to pass through the essentially tubular body passage.

51. The method of claim 50, further comprising the steps of monitoring the tension in the lengthening device and manipulating the device to increase the force or moving the device to a new location.

52. The method of claim 50, further comprising the steps of monitoring the lengthening device with an endoscope.

53. The method of claim 50, further comprising the steps of monitoring the lengthening device with a method for navigating through an organ via a computer generated image of the organ.

54. The method of claim 50, further comprising the steps of inserting and deploying a multiplicity of lengthening devices simultaneously in a patient, thereby allowing for a rapid increase in the total essentially tubular body passage length.
